(19) United States
(12) Patent Application Publication
(10) Pub. No.: US 2013/0096029 A1
(43) Pub. Date: Apr. 18, 2013

(54) MULTIPLEXED IN VIVO SCREENING OF BIOLOGICAL SAMPLES

(71) Applicants: Samuel K. Sia, New York, NY (US); HESAM PARSA, New York, NY (US)

(72) Inventors: Samuel K. Sia, New York, NY (US); HESAM PARSA, New York, NY (US)

(73) Assignee: THE TRUSTEES OF COLUMBIA UNIVERSITY IN THE CITY OF NEW YORK, New York, NY (US)

(21) Appl. No.: 13/605,878

(22) Filed: Sep. 6, 2012

Related U.S. Application Data
(60) Provisional application No. 61/531,573, filed on Sep. 6, 2011.

(51) Int. Cl. C12Q 1/02

(52) U.S. Cl. CPC C12Q 1/025
USPC 506/10; 506/39; 506/14

(57) ABSTRACT

Microfabricated platforms can be used to study a heterogeneous panel of biosamples in a realistic in vivo setting. The platform can be formed of a polymer (e.g., a hydrogel) and can be constructed for implantation into an animal host for in vivo testing. The platform can have a plurality of testing regions therein that are constructed to allow exposure of the testing region to the host stroma when implanted in vivo. For example, the microfabricated platform can be used for screening different cancer cell-lines (e.g., to identify which cell line responds to an anti-cancer drug) or for screening different biomaterials (e.g., to identify a composition with ideal host response for a specific implantable device).

### CROSS-REFERENCE TO RELATED APPLICATIONS

The present application claims the benefit of U.S. Provisional Application No. 61/531,573, filed Sep. 6, 2011, which is hereby incorporated by reference herein in its entirety.

### FIELD

The present application relates generally to testing of biological samples, and, more particularly, to systems, methods, and devices for multiplexed in vivo screening of biological samples, for example, for cancer drug screening or material biocompatibility testing.

### BACKGROUND

Monolayer culture systems are used by pharmaceuticals and research labs to investigate the efficacy of anti-cancer therapeutic agents. However, the inability of the monolayer system to mimic tumor microenvironment leads to inaccurate prediction of drug efficacy in vivo. Often, promising lead compounds fail in the later phases of clinical trials despite initially encouraging results. Compared to other therapeutic areas, there is a particularly high attrition rate for anti-cancer drugs. Indeed, in recent years there have been a large number of unsuccessful clinical trials, with only 8% of drug candidates which enter Phase I trials actually reaching the bedside.

Individual anti-cancer compounds may only be effective for cells with specific genotypes. Efficacy studies to determine which genotypes a particular drug is effective against are performed at a relatively low throughput in animal models. A single compound is thus screened against one genotype per animal. While animal models, such as transgenic mice and xenograft models, can serve as a promising tool for preclinical studies, the low throughput of these systems and the inability to test large number of cell-lines limits their potential to capture the genomic heterogeneity of cancer. It is therefore logistically challenging to identify the subgroups of responsive cancer cells for the developed rationally targeted drugs.

### SUMMARY

An effective high-throughput method to identify in vivo (not just in vitro) efficacious compounds, as well as identifying the cell types that would be susceptible to compounds and/or other treatment therapies or devices, well before the expensive Phase II/III clinical trials could make the drug discovery process much more cost-effective. In addition, biomaterials can be used in various in vivo applications, such as drug delivery devices, artificial heart valves, intraocular lenses, scaffolds for cell transplantations, coatings for medical implants, etc. The success of these applications relies at least in part on the response of the host to the implanted biomaterial. The testing of host response can be performed by implanting a material of a single composition into a single host. In order to test the biocompatibility of many compositions and/or materials, many animals would be necessary. Not only would a large number of animals need to be sacrificed during such testing, but variance in observed responses due to inconsistencies in host microenvironment and in surgical operation may increase. High throughput testing of in vivo host response (i.e., biocompatibility) of biomaterials may serve to reduce the number of animals needed as well as improve testing results and consistency.

Microfabricated platforms can be used to study a heterogeneous panel of biosamples in a realistic in vivo setting. The platform can be formed of a polymer (e.g., a hydrogel) and can be constructed for implantation into an animal host for in vivo testing. The platform can have a plurality of testing regions therein that are constructed to allow exposure of the testing region to the host stroma when implanted in vivo. For example, the microfabricated platform can be used for screening different cancer cell-lines (e.g., to identify which cell line responds to an anti-cancer drug) or for screening different biomaterials (e.g., to identify a composition with ideal host response for a specific implantable device). For example, hydrogel precursors and cells may be 3D-printed to form a structure that immobilizes the cells. Alternatively, cells may be sandwiched between preformed hydrogel plates. Still other means for fabricating three-dimensional or two-dimensional structures can be employed. Any structure suitable for immobilizing and isolating cells of differing genotype (or other characteristic) from each other while permitting the transmission of mechanical, chemical, thermal, or other signals from stromal cells may be employed.

In one or more embodiments, the testing regions may be individual chambers holding respective tumor spheroids therein. A membrane layer may retain the tumor spheroid within the interior of each chamber while allowing stromal cell interaction with the tumor spheroid. The tumors may be of different genotypes from each other so as to allow simultaneous testing of multiple genotypes in a single host animal. An anti-cancer drug (or any other type of therapeutic device or agent) can be given to the host animal while (or before) the platform is implanted therein, thereby subjecting each of the tumor genotypes to the same host environment at the same time. The construction and arrangement of the platform and the testing regions may be such that the cross-talk between adjacent testing regions (e.g., unintended or undesirable interactions between tumor cells of different genotypes) is minimized or at least reduced.

In one or more embodiments, the testing regions may be individual biomaterials formed on or in the platform. A surface of the individual biomaterials can be exposed to the host stroma for interaction therewith. The individual biomaterials can have different compositions and/or materials from each other so as to allow simultaneous testing of multiple types in a single host animal. The platform can be implanted in the host animal and left to interact with the host for a period of time. Biocompatibility (or other desired characteristics of the implanted biomaterials) can be ascertained for each biomaterial by appropriate inspection and analysis of the extracted platform.

In one or more embodiments, a method of screening multiple biological samples in vivo can include providing a platform (e.g., of polymer) having a plurality of testing regions thereon. A separate one of the biological samples can be placed in each of the testing regions. The method can further include implanting the platform into a host animal such that each of the biological samples interacts with stroma cells of the host animal. The method can also include removing the platform from the host animal to evaluate said testing regions. The method can further include, after the implanting, administering a cancer drug to the host animal.

The testing regions can be arrayed in two dimensions across the platform, for example, as a circular or rectangular array. The number of testing regions on the platform can be at least 20. The testing regions can be configured and arranged such that cross-talk between the biological samples while implanted in the host animal is prevented or at least reduced. The biological samples can be tumor spheroids. Each tumor spheroid can be a different genotype. Alternatively or additionally, the biological samples can be materials for biocompatibility testing. For example, the materials can include hydrogels.

The platform can include a plurality of microfluidic channels. Each of the microfluidic channels can be connected to a respective one of the testing regions. Placing the platform can include flowing cancer cells through the microfluidic channels to load each chamber and forming in each chamber a tumor spheroid from the cancer cells therein. Each chamber can include a membrane layer with pores therein. The membrane layer can be constructed to retain the tumor spheroid in the chamber. The pores can be sized and shaped to allow infiltration of host animal stroma cells after the implanting. The chamber can have a diameter of 500 μm or less and a height of 300 μm or less. The membrane layer can have a thickness of 20 μm or more.

In one or more embodiments of the disclosed subject matter, a device for screening multiple biological samples in vivo can include a platform member. The platform member can have a plurality of testing regions thereon. Each of the testing regions can be configured to hold a different biological sample for interaction in vivo with stroma cells when implanted into a host animal. The platform can also include isolating portions arranged between adjacent testing regions such that fluid and/or material cannot pass from one testing region to another testing region without contacting the host animal stroma cells when implanted into the host animal.

The testing regions can be arranged in a two-dimensional array with isolating portions arranged therebetween. The testing regions and isolating portions can be arranged such that crosstalk between testing regions is inhibited or at least reduced. For example, the isolating portions can protrude from a planar surface of the platform. Each testing region can include a chamber with a membrane layer. The membrane layer can be constructed to retain a tumor spheroid within the chamber while allowing interaction between the tumor spheroid and host cell stroma through the membrane layer. The platform can include a plurality of separate microfluidic channels. Each channel can connected to a respective chamber. Alternatively or additionally, each testing region can include a block of a biomaterial to be tested and the isolating portions can include a biocompatible hydrogel-based backing layer.

In one or more embodiments, a system for screening multiple biological samples in vivo can include a screening device and an evaluation device. The screening device can include a platform (e.g., of polymer) with a plurality of testing regions thereon. Each of the testing regions can be configured to hold a different biological sample for interaction in vivo with stroma cells when implanted into a host animal. The platform can further include isolating portions arranged between adjacent testing regions such that fluid and/or material can only pass from one testing region to another testing region by contacting the host animal stroma cells when implanted into the host animal.

The evaluation device can be configured to image the testing regions ex vivo so as to determine the effect of the in vivo exposure on the biological samples in the platform. The evaluation device can include an imaging device configured to acquire an image of each biological sample. The platform can be constructed such that the biological samples can be imaged by the imaging device in situ. The biological samples can include biomaterials for biocompatibility testing. The evaluation device can include a processor configured to determine inflammatory cell density on each biomaterial based on the images from the imaging device. Alternatively or additionally, the biological samples can include tumor spheroids of different genotypes, and the evaluation device can include a processor configured to determine for each tumor spheroid at least one of spheroid diameter, change in spheroid size, viable cell mass, percentage viability, and pathway activity based on the images from the imaging device.

In one or more embodiments, a method of culturing biological samples can include arranging biologically varied cells or tissue cultures in an array that spans a curvilinear planar region and exposing the varied cells or tissue cultures to living tissue of a host animal such that the cells or tissue cultures receive chemical signals from the living tissue. The method can further include regulating or perturbing the natural state of the host, and detecting an effect of the regulating or perturbing on the cells or tissue cultures. For example, the regulating or perturbing can include delivering a drug to the host animal. The curvilinear planar region can be a substantially flat planar region.

The arranging can include placing the varied cells or tissue cultures in a unitary structure having a respective compartment for each of the biologically varied cells or tissue cultures. Alternatively or additionally, the arranging can be such that an exposed face of the cells or tissue cultures face in a same direction.

The unitary structure can be configured to space the cells or tissue cultures less than 5 mm apart, for example, less than 1 mm apart. The unitary structure can have at least 20 compartments. The unitary structure can include microfluidic channels, each of which is connected to a respective one of the compartments and configured for loading the compartment. Each compartment of the unitary structure can have a diameter of 500 μm or less and a height of 300 μm or less. Each compartment can be isolated from the host animal by a membrane. Each membrane can be porous with a pore size to exclude the transfer of cytoplasmic bodies thereacross.

The exposing can include permitting the transmission of chemical signals across a membrane. Alternatively or additionally, the exposing can include surgically implanting the unitary structure in the host animal, for example, by surgically implanting the unitary structure in a stromal compartment of an organ of the host animal. The detecting can include surgically removing the unitary structure and performing an assay on the cells or tissue cultures in vitro while they are in the unitary structure.

In one or more embodiments of the disclosed subject matter, a method of performing primary efficacy screening of a treatment device against a number of genotypes can include selecting a candidate treatment device applicable to an animal and applying the treatment device to the animal. The method can further include implanting cancer cells of a variety of genotypes in the host and culturing the cancer cells for a period of time. The effects of the treatment device on the host can be measured as well as its effects on the implanted cancer cells. The method can further include storing data, in a data storage device, of identifiers of each of the genotypes against the corresponding measured effect of the treatment device.

The implanting can include implanting a unitary structure that immobilizes the cancer cells. The measuring can include removing the unitary structure that immobilizes the cancer cells. Alternatively or additionally, the measuring can include assaying the cancer cells within the unitary structure. The treatment device can include the administration of a compound to the blood of the animal. The culturing can include providing nutrients to the cancer cells from the animal.

Objects and advantages of embodiments of the disclosed subject matter will become apparent from the following description when considered in conjunction with the accompanying drawings.

### BRIEF DESCRIPTION OF DRAWINGS

Embodiments will hereinafter be described with reference to the accompanying drawings, which have not necessarily been drawn to scale. Where applicable, some features have not been illustrated to assist in the illustration and description of underlying features. Throughout the figures, like reference numerals denote like elements.

FIG. 1 is a schematic diagram showing a plan view of a platform for multiplexed in vivo screening, according to embodiments of the disclosed subject matter.

FIG. 2 is a schematic diagram showing a cross-sectional view of a platform with cells or tissue cultures in each testing region, according to embodiments of the disclosed subject matter.

FIG. 3 is a schematic diagram showing a cross-sectional view of a platform for cell/tissue culture testing with a microfluidic channel, according to embodiments of the disclosed subject matter.

FIGS. 4A-4B are images of pores in the membrane layer of a platform, according to embodiments of the disclosed subject matter.

FIG. 5 is a schematic diagram showing a layout for a circular array of testing regions and separate microfluidic channels, according to embodiments of the disclosed subject matter. The inset at the bottom of the figure shows a close-up view of some of the testing regions.

FIG. 6 shows images of a fabricated testing region, according to embodiments of the disclosed subject matter.

FIG. 7 is a schematic illustration of the formation of a tumor spheroid in a testing region chamber of a platform, according to embodiments of the disclosed subject matter.

FIG. 8 is a schematic diagram showing a cross-sectional view of a platform with biomaterials in each testing region, according to embodiments of the disclosed subject matter.

FIGS. 9A-9B are diagrams showing isometric and cross-sectional views, respectively, of microfluidic fabrication of an array of biomaterials for testing, according to embodiments of the disclosed subject matter.

FIGS. 9C-9D are diagrams showing isometric and cross-sectional views, respectively, of encapsulation of the array of biomaterials in a backing layer, according to embodiments of the disclosed subject matter.

FIG. 9E is a diagram showing a finished platform with an array of biomaterials, according to embodiments of the disclosed subject matter.

FIG. 9F is a diagram showing a cross-sectional view of a platform with an array of biomaterials, according to embodiments of the disclosed subject matter.

FIG. 10 is a process flow diagram for use of the platform for multiplexed screening of biological samples, according to embodiments of the disclosed subject matter.

FIG. 11 is a simplified schematic diagram of a system for multiplexed screening multiple biological samples, according to embodiments of the disclosed subject matter.

### DETAILED DESCRIPTION

Embodiments of the disclosed subject matter include systems, methods, and devices for high throughput multiplexed in vivo screening of biological samples. A platform can be microfabricated (e.g., by or including microfluidic techniques) for implantation into a live host animal (e.g., an animal model) so as to allow for simultaneous screening of a relatively large number (i.e., at least twenty) of biological samples carried by the platform in a realistic and heterogeneous microenvironment that resembles the tumor stroma. The biological samples can include cells, tissue cultures, and/or biomaterials. For example, the biological samples can include different genotypes of cells susceptible to cancer drugs (e.g., tumor spheroids) and/or different biomaterials that elicit different host responses (e.g., hydrogels to be used for implantable devices). For example, embodiments of the disclosed platform can be used to compare the statistical efficacy of anti-Hh therapeutics (GDC-0449, LDE225, BMS-833923, IPI-926, PF-04449913, LEQ506 and TAK-44, currently in clinical trials) over a heterogeneous panel of medulloblastoma cancer cells in vivo or to study the host response of a hydrogel for use in drug delivery or tissue engineering.

Multicellular tumor cell spheroids (MTCS) are believed to accurately emulate tumor cells in vivo both in terms of their pathophysiology and response to therapy. These aggregates can mimic tumor tissue more effectively than regular 2D cell cultures because spheroids, much like tumors, usually contain both surface-exposed and deeply buried cells, proliferating and non-proliferating cells, and well-oxygenated and hypoxic cells (the latter secreting tumor cell cytokines). Nevertheless, it remains extremely difficult, if not impossible, to recreate tumor microenvironment in vitro. Implanting these MTCS using the disclosed platform into a live host animal (such as transgenic mice or xenografts) treated with a particular anti-cancer drug can allow the identification of responsive genotypes that warrant further exploration via human trials. Thus, a genotype-by-genotype analysis of which tumor cells may be responsive to a particular drug candidate can be generated.

Depending on the specific application, the platform can be fabricated from a wide range of biomaterials, such as, but not limited to, elastomeric-based polymers (e.g., polydimethylsiloxane (PDMS)) and/or photocrosslinkable hydrogels (e.g., PEGylated fibrinogen). For example, the platform can be a multi-layer construct that includes at least a supporting layer and an array of multiplexed biological samples for testing.

When dealing with biological samples that are not photocrosslinkable, soft lithography techniques can be used to form the platform with appropriate receptacles therein for receiving the biological samples. For example, microfabrication techniques such as those disclosed in U.S. Publication No. 2011/0015739, entitled “Systems and Methods for Forming Patterned Extracellular Matrix Materials,” which is hereby incorporated by reference herein in its entireties, can be used to form the platform or other components used to form the platform. Using the techniques disclosed in the '739 publication, a stamp can be used to form receptacles in a first polymer layer for the biological samples. After inserting the samples, the receptacles can be sealed by providing a second polymer layer thereover, although the second polymer layer may include openings or pores to allow communication between the samples and the host environment. Other soft lithography techniques are also possible according to one or more contemplated embodiments.

When dealing with biological samples that are photocrosslinkable, microfluidic-based photopolymerization can be used to fabricate the platform with the biological samples (e.g., materials for biocompatibility testing) provided therein. For example, microfabrication techniques, such as those disclosed in U.S. Publication No. 2008/0286482, entitled “Forming or Patterning Composite Microstructures Using Microfluidics,” and in U.S. Publication No. 2010/0278798, entitled “Methods and Systems for Forming Biocompatible Materials,” which are hereby incorporated by reference herein in its entirety, can be used to form the platform or other components used to form the platform. Using the techniques disclosed in the '842 publication, photopolymerizable biosamples can be provided in a microfluidic channel and selectively polymerized in situ by light application (e.g., via a laser). Unpolymerized biosamples can be removed and different biosamples can be flowed in and polymerized in order to form an array of biosamples for testing. A separate backing layer can be provided to support the biosample array for implantation.

Using the techniques disclosed herein, a genetically heterogeneous panel of cancer cell lines (or any other cell line or tissue) and/or a wide range of biomaterials (or any other material) can be micropatterned into a millimeter-sized cassette. Such a platform reflects the heterogeneity of the biological systems (such as genomic heterogeneity of a cancer and versatile properties of biomaterials), which may facilitate the assessment of drug or treatment efficacy and biomaterial host response in a realistic in vivo setting with minimal number of animals. By reducing the number of animals required, this approach can potentially reduce the cost and time of the labor-intensive in vivo testing phase, reduce animal-to-animal variation in the material screening process, and improve conformity with the bioethics of animal testing.

The platform can be implanted subcutaneously in an animal host (e.g., a mouse or other animal model) followed by customized treatment (e.g., administration of a cancer drug). After the extraction, each of the biological samples can be characterized in a high content fashion. For example, each of the biological samples may be imaged and/or processed to determine tumor spheroid size, necrotic cell distribution, pathway activities, and/or inflammatory cell density before and/or after implantation.

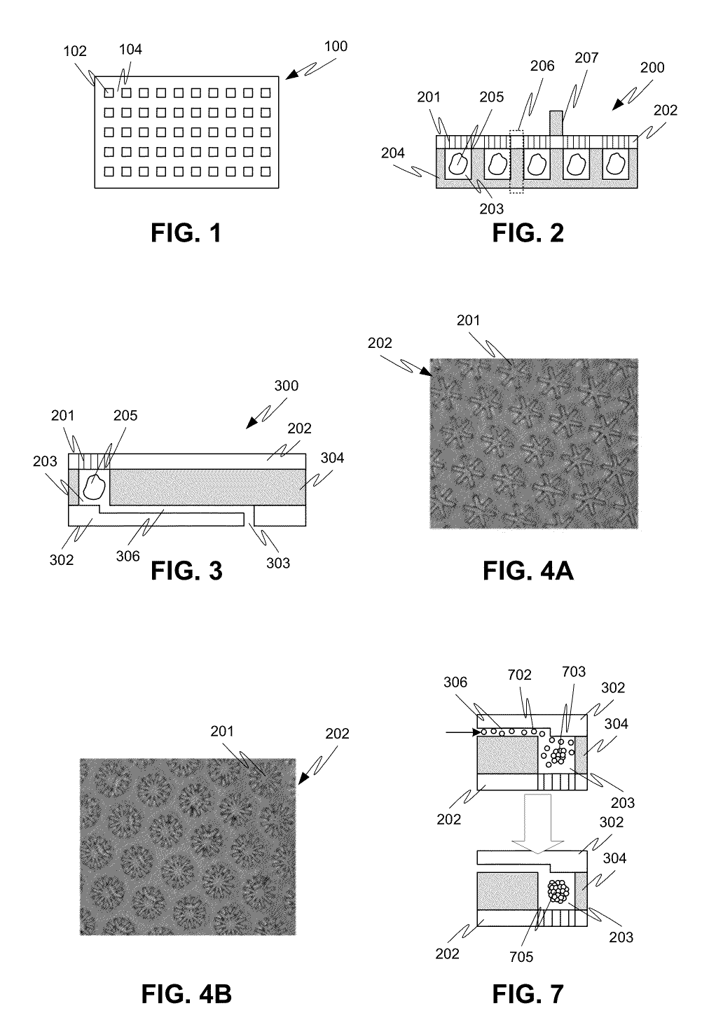

Referring to FIG. 1, a microfabricated platform 100 is shown. The platform 100 can be formed of a polymer, for example, PDMS. Other materials for the platform 100 can also be used according to one or more contemplated embodiments. For example, the platform may be formed of polyethylene glycol diacrylate (PEGDA). In embodiments, the platform is an engineered tissue or tissue precursor of hydrogel that immobilizes cell-containing portions indicated by 102. In other embodiments, discussed in more detail below, the platform is any kind of monolithic microfluidic member that may be made of plastic, ceramic, or other materials.

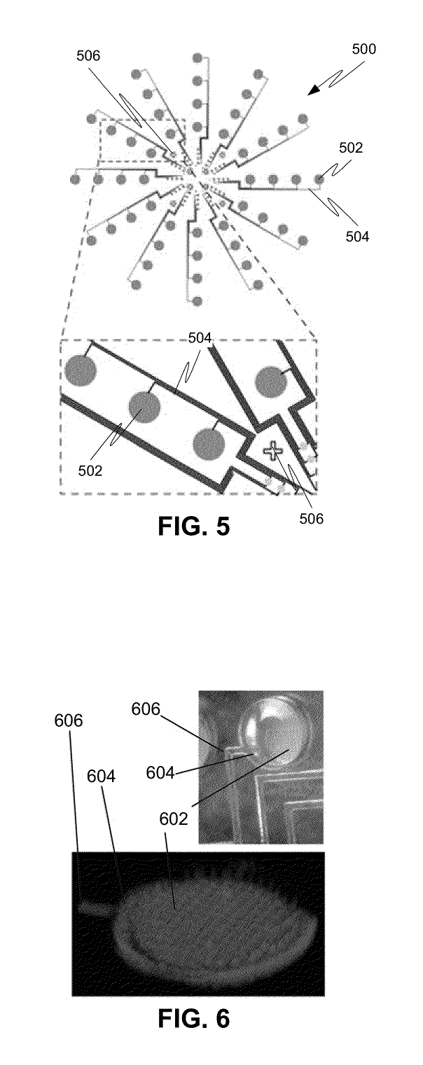

Each testing region 102 can include a biological sample for testing in vivo. The biological sample in each testing region can be different from each other (i.e., to allow simultaneous multiplexed screening) or the same (i.e., to allow simultaneous redundant screening). Although the testing regions 102 are shown as a rectangular array in the figure, the testing regions 102 can be arranged in other regular or irregular arrays, such as a circular array (see, for example, FIG. 5), or randomly. In addition, although the platform 100 is illustrated as having a rectangular shape, it may take other shapes, such as circular or elliptical forms.

Individual testing regions 102 can be arranged such that cross-talk or interference between the different biological samples during in vivo implantation is prevented or at least reduced. Portions 104 of the platform can be arranged between the individual testing regions 102 that prevent cross-talk. Such portions 104 are referred to herein as isolating portions.

The isolating portions 104 can be a portion of a supporting substrate that extends between the testing regions 102 and/or a wall of a receptacle in the testing region 102 for the biological sample. The isolating portion 104 thus forces fluid and/or materials to interact with host animal stroma in traveling between testing regions 102. In other words, there is no direct flow path between adjacent testing regions 102 that does not contact the host animal stroma when the platform 100 is implanted into the host animal.

Referring to FIG. 2, a cross-sectional view of a platform 200 for screening a biological sample 205 is shown. Each biological sample can be a different cell or tissue culture, or a material to be tested. Platform 200 can be a multi-layer construct containing the sample 205 therein. A first layer 204 can include individual testing regions formed as chambers or compartments 203 for holding the samples 205. The samples 205 can be retained in the chambers 203 by a second layer 202. The second layer 202 can be attached to the first layer 204 and sealed thereto such that the samples 205 cannot escape from the chambers 203 when the platform 200 is implanted into the host animal. Isolating portion 206 separates adjacent chambers 203 from each other such that crosstalk is minimized, or at least reduced. Additional isolating portions, such as protrusion 207 which extends from a surface of the first layer 204, can be provided to increase the length of the flowpath between adjacent chambers 203 to further reduce the possibility of crosstalk.

Communication between the samples 205 and the host animal microenvironment is allowed via pores or openings 201 in the second layer 202. For example, when dealing with cells as the biological sample 205, the pores may have a size of approximately 4 μm to prevent passage of the cells through the second layer 202 while still allowing chemical signals to pass between the cells and the host animal microenvironment. The size and shape of the pores can be designed for adequate adhesion between the first and second layers while still preventing egress of the cells (or other samples) from the chamber. For example, the pores can be shaped as shown in FIGS. 4A-4B.

Only five chambers are illustrated in FIG. 2, but embodiments of the disclosed subject matter are not limited thereto. Rather, the chambers can be arrayed (regular or irregular) in any dimension to achieve a desired number of chambers for testing. For example, at least 20 chambers are provided in a 2D array for testing of biological samples. Each chamber can be a cylinder with a diameter of less than 500 μm, for example, 400 μm. The height of each chamber can be less than 300 μm, for example, 200 μm. The distance between adjacent chambers can be at least 800 μm. The thickness of the second layer 202 can be less than 20 μm and the overall thickness of the platform 202 can be less than 1 mm. The dimensions of the platform 202 in plan view can be on the order of millimeters, for example, 5 mm or less. The above noted dimensions are exemplary in nature, and other dimensions are also possible according to one or more contemplated embodiments.

In order to load each chamber 203, the sample 205 can be inserted into the chamber 203 prior to joining the second layer 202 to the first layer 204. Alternatively, the chamber 203 can be loaded with the sample 205 (e.g., tumor cells) via a corresponding microfluidic channel. For example, a third layer 302 can be provided to a first layer 304 as shown in FIG. 3. The first layer 304 may form part of the chamber 203 with portions of the first layer 202 and the third layer 302 forming other parts of the chamber 203. The third layer 302 may have a microfluidic channel 306, such that fluids and/or materials can be provided to the chamber 203 by injecting into inlet 303.

Thus, the platform can include, for example, three polymer layers: a loading layer 302, a chamber layer 304, and a membrane layer 202. Although FIG. 3 only shows a single chamber 203 and channel 306, a practical embodiment would include multiple chambers with respective channels. As shown in FIG. 5, for example, a plurality of individually addressable chambers 502 can be provided for holding a unique biological sample to be tested. Each chamber 502 may have a corresponding microfluidic channel 506 connected thereto such that each chamber 502 can be independently loaded. For example, the number of chambers may be greater than 20, such as the 48 chambers shown in FIG. 5. However, greater or fewer chambers are also possible according to one or more contemplated embodiments. For example, the chambers may number in the hundreds (e.g., such as 386-well array). Each biological sample may be a different cell or tissue culture, such as a tumor spheroid of a unique cancer genotype.

The platform 500 shown in FIG. 5 can be created using soft lithography techniques, for example. As discussed above with respect to FIG. 3, platform 500 can have three layers—a loading layer, a chamber layer, and a membrane layer. The membrane layer, which acts as a barrier between the host and the biological sample encapsulated within each chamber 502, can be a 20 μm-thick perforated PDMS layer (e.g., with an array of 40 μm diameter holes). The chamber layer, which holds forty-eight 400 μm diameter, 200 μm tall cylindrical microchambers 502, can be a 200 μm PDMS layer sandwiched between the loading and membrane layers. The loading layer can be a thick PDMS layer used to load the biological sample within the microchambers 502 via 100 μm-wide, 100 μm-tall microchannels 504. Note that each of the microchannels may be sealed from the others and associated with a particular microchamber such that the microchambers may be independently loaded without potential for cross-contamination. In addition, outlet channels can be provided as well as inlet channels in order to convey fluid/materials from the microchambers 502.

The layers of platform 500 may be aligned using any suitable device and cured together to form a single unitary device. For example, an alignment device that employs metal guides and fixed-alignment bearings can be used to eliminate lateral motion when aligning the layers together. Alignment marks 506 can be provided on each layer and used to visually align the layers prior to bonding. Once aligned, the PDMS pieces can be cured at 70° C. to form a hermetic seal

When dealing with cells as the biological material, pore sizes in the membrane layer should be relatively small (e.g., ˜4 μm). However, due to large thickness of the posts, the aspect ratio of these structures is relatively large, which may result in fragile structures, weak adhesion, and/or other problems during the fabrication process. Membrane designs such as those illustrated in FIGS. 4A-4B may maximize or at least improve the adhesion strength of the posts while allowing the width to be sufficiently small in order to trap the cells. An image of a fabricated chamber 604 with membrane 602 and input channel 606 is shown in FIG. 6.

Referring to FIG. 7, each chamber 203 in the platform can be loaded with cells via the respective microchannel 306. When dealing with cancer cells 702, the cells can be cultured within the chamber 203 to form an MTCS in each chamber 203. For example, cancer cells 702, such as cancer epithelial cells or HT-29 and DLD-1 colon cancer cell lines, can be flowed into each chamber 203, with each chamber receiving a distinct cell genotype. The cells in the chamber 203 can be in culture media and can begin to form an MTCS 703. After culturing (e.g., 1-3 days), an MTCS 705 may be formed in the chamber 203. For example, a defined number of cells ranging from 1,000 to 2,000 can be loaded into the chambers in supplemented RPMI 1640 media, centrifuged for 3 minutes at 500 g, and cultured for 24 hours to produce single spheroids in each chamber with homogeneous sizes.

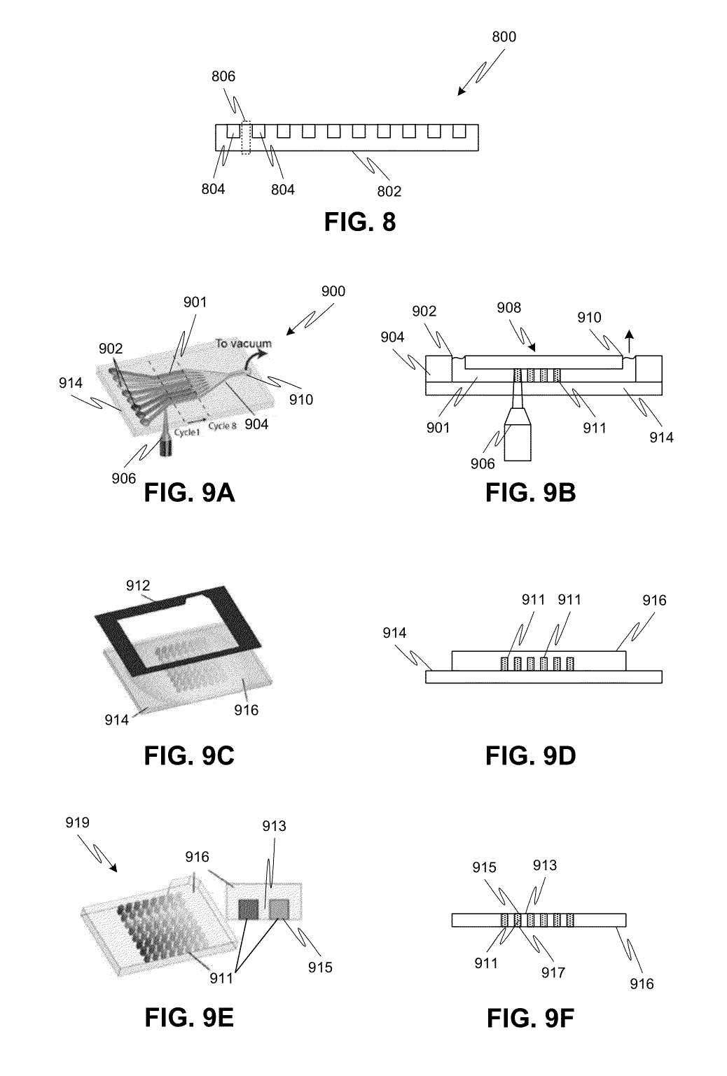

Referring to FIG. 8, a platform 800 for testing host remodeling of biomaterials is shown. The platform 800 includes a plurality of testing regions 804. Each testing region 804 can be a different biomaterial for testing, for example, a different hydrogel composition for biocompatibility testing. A backing layer 802 supports the individual testing regions 804 therein. Portions 806 of the backing layer 802 between adjacent testing regions 804 are isolating regions, similar to isolating regions 206 described above with respect to FIG. 2. By implanting platform 800, the biological response to a plurality of different material compositions (e.g., 48 different hydrogel compositions) can be simultaneously evaluated in a single animal.

The biological samples in each testing region 804 and/or the backing layer 806 can be formed of a hydrogel. Thus, the implantability and biodegradability of the whole platform 800 (including the hydrogel backing material 806) may make the platform 800 suitable for multiplexed in vivo screening. For example, the implantable platform 800 can be a 6-by-8 array of 48 hydrogel microstructures that are mixtures of synthetic and natural extracellular matrix components (e.g., PEGDA). The entire hydrogel microarray can be easily implanted into animals subcutaneously and retrieved to evaluate host response with histological tools. Thus, in a single animal, the host response to a variety of biomaterial compositions and 3D microenvironments can be simultaneously observed, providing an initial screening of the desired properties.

A biodegradable hydrogel, PEGylated fibrinogen (PEG-fibrinogen) can be used as a base material and can be supplemented with a synthetic hydrogel precursor, PEGDA. The denatured fibrinogen fragments can form the backbone of a PEG-fibrinogen/PEGDA hydrogel network and can provide adhesion and protease degradation sites. The structural properties of the hydrogel, such as pore size and permeability, can be changed by adding different amount or types of PEGDA precursors. For example, the concentration (1-5 w/v%) and chain length (400 Da, 4 kDa, and 10 kDa) of the additional PEGDA constituent can be varied to form 48 different hydrogel compositions and evaluate their host responses simultaneously. Host responses can be evaluated in terms of the degree of inflammatory cell adhesion (e.g., inflammatory cell density) as well as infiltration and hydrogel degradation.

Referring to FIGS. 9A-9E, a fabrication method for a platform for testing biological materials is shown. Each platform can have, for example, 48 hydrogel blocks 911 in a 6-by-8 array format, with each hydrogel block representing a unique hydrogel composition. Each biological material block 911 may be in the form of a cube, for example, having a dimension of 400 μm. However, other shapes and sizes for each biological material block 911 are also possible according to one or more contemplated embodiments.

To fabricate the platform, different hydrogel prepolymers can be provided to separate inlets 902 and flowed into respective microchannels 901 on a microfluidic chip 900, as shown in FIGS. 9A-9B. The microfluidic chip 900 can have a microchannel layer 904 (e.g., a PDMS layer) on a support 914 (e.g., glass layer) that forms parallel microchannels 901 which converge at a common outlet 910. The chip 900 can be mounted over an illuminating light source 906, for example, an epifluorescence microscope equipped with a motorized stage. During each cycle, approximately 20 μL of 8 different prepolymers can be applied at the inlets 902 and flowed into each channel 901 simultaneously by applying negative pressure at the outlet 910. Once the flow had stopped, UV light from source 906 can be used to expose the fluid in each channel sequentially in one direction through a rectangular field diaphragm (e.g., 400 μm×600 μm) for approximately 20 to 40 s.

The prepolymer in each channel 901 is thus photopolymerized into a hydrogel block 911 that adheres to the bottom of the channel 901 and is aligned with the hydrogel blocks 911 in the other channels 901 in the scanning direction. The channels can then be purged with a series of PBS washes to remove any unpolymerized materials. Another 8 prepolymers can be introduced into the microchannels 901, and the cycle repeated to generate additional hydrogel blocks 911 within array region 908.

Upon completion of the last cycle, the microchannel layer 904 of the microfluidic chip 900 can be removed as shown in FIGS. 9C-9D. The hydrogel blocks 911 can be covered with a plain hydrogel prepolymer, for example, 400 μL of plain hydrogel prepolymer (10 w/v% PEGDA, M.W.: 10 KDa). A transparency mask 912 can be used to expose the hydrogel prepolymer to a UV flood lamp, for example, for 15 s at ˜20 mW/cm². Polymerization of this prepolymer by the UV exposure forms backing layer 916 with the array of hydrogel blocks 911 contained therein.

The backing layer and array of hydrogel blocks can then be removed from the support 914 to provide the microfabricated platform 919, as shown in FIG. 9E. For example, platform 919 can have a thickness of 0.1 cm, a width of 1 cm, and a length of 1.5 cm. Each of the hydrogel blocks 911 thus has a top surface 915 that will be exposed to host stroma cells upon implantation, while other surfaces are isolated from adjacent blocks 911 as well as the host stroma cells by isolating portions 913 of the backing layer 916. The exposed surface 915 of the blocks 911 may all face in the same direction, as shown in FIGS. 8 and 9E.

Alternatively, the exposed surface 915 of adjacent blocks 911 may face in opposite directions. For example, a first block 911 may be exposed only at a top surface of the backing layer 916, while blocks 911 adjacent said first block are only exposed at a bottom surface of the backing layer 916. In yet another alternative, each block 911 may have a first surface 915 exposed at a top surface of the backing layer 916 and a second surface 917 exposed at the bottom surface of the backing layer 916, as shown in FIG. 9F.

Inflammatory cell infiltration to the site of implant occurs in response to cell-derived cytokines and chemokines and also growth factors generated as a result of the activation of the inflammatory response. Due to the presence of these soluble factors, host responses to hydrogel blocks 911 in close vicinity may result in crosstalk effect. The order of hydrogel blocks 911 in the array can be switched to be used as control samples. Alternatively or additionally, other measures may be used to help control crosstalk, such as, but not limited to, arranging exposed surfaces of adjacent blocks 911 to be on opposite sides of the backing layer 916, increasing the distance between adjacent blocks 911 and thus the size of isolating portions 913, and providing additional isolating portions (e.g., similar to protrusion 207 in FIG. 2).

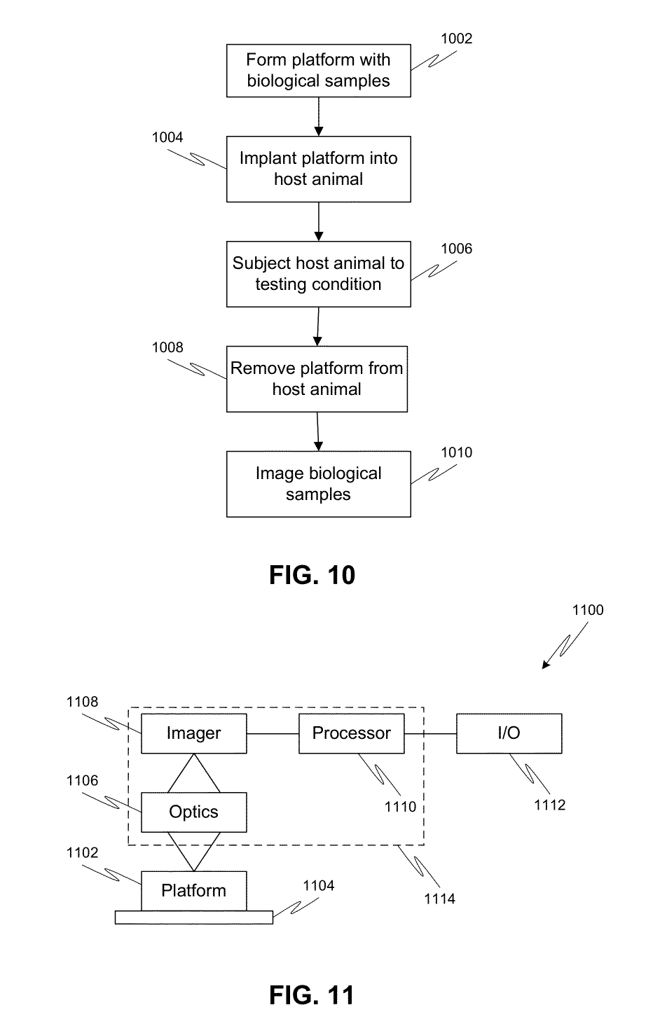

Referring to FIG. 10, a method of screening multiple biological samples is shown. At 1002, a platform can be formed with the biological samples for testing. The platform can be one or more of those discussed with respect FIGS. 1-9F above and formed according to the procedures disclosed elsewhere herein. At 1004, the formed platform can be implanted into a host animal for testing. The implantation may be such that the biological samples in the platform are exposed to the living tissue of the host animal and such that the samples can receive chemical signals from the living tissue. For example, the platform can be surgically implanted into a mouse model subcutaneously such that the biological samples are exposed to the mouse stroma cells.

At 1006, the host animal can be subject to one or more test conditions. For example, when the biological samples are cancer cells, the host animal may be subject to a cancer drug treatment or other therapeutic for a predetermined period of time (e.g., for three days). Such test conditions may serve to regulate or perturb the natural state of the host animal. When the biological samples are materials for biocompatibility testing, the host animal may be allowed to engage in normal behavior or be subject to other conditions that may impact the biocompatibility results (e.g., diet, exercise, drug administration, or any other condition). The platform may remain within the animal host for a predetermined period of time (e.g., for three days).

At 1008, the platform can be removed from the host animal for evaluation. For example, the platform can be surgically removed with or without killing the host animal. At 1010, biological samples can be imaged for evaluation. For example, when the biological samples are cancer cells, the tumor spheroids formed by the cancer cells can be characterized in situ simultaneously for all chambers of the platform. The tumor spheroids may be characterized according to, for example, change in size, as determined from an image of the platform and the spheroids therein. When the biological samples are materials for biocompatibility testing, the materials can be characterized in situ according to, for example, inflammatory cell density on the exposed surface of each material, as determined from an image of the platform and the exposed material surfaces. Imaging and evaluation of the platform can be performed, for example, using a system as shown in FIG. 11 and described in more detail below.

For example, tumor cells can be stained and imaged for proliferation, caspase-3 activity and necrosis. Proliferating cells in the platform post-implantation can be detected with a cell proliferation fluorescence kit. Caspase-3 activity and necrosis in cancer cells can be with Caspase-3 assay kit for live cells and propidium iodide probe, respectively. Imaging of the cells after staining can be done using a microscope, such as a confocal microscope system, a fluorescence microscope, or any other optical imaging technique. The results can be quantified as the percentage of caspase-3/PI positive cells in the population.

In another example, inflammatory response of a particular biomaterial can be characterized by histological staining Excised platforms can be rinsed with PBS, fixed in buffered, neutral 10% formalin solution overnight, embedded in paraffin, and sectioned in the transverse plane and parallel to the long axis of the hydrogel platform at 5 μm thickness according to standard histological procedures. Sequential sections can be stained with standard hematoxylin and eosin (H&E) dye. For each biomaterial composition, cross-sections of each biomaterial block can be examined, for example, using light microscope to obtain the inflammatory cell (i.e., monocytes, macrophages, giant cells) density. Inflammatory cell density can be determined by counting the number of inflammatory cells, normalized by the area of interest. A processor can be used to determine cell count and cross-section area from an obtained image.

After the 1st week of implantation, there may be different degrees of adhesion of inflammatory cells, including neutrophils, monocytes, and macrophages, on each hydrogel block of the implanted platform. Since the backing hydrogel should be chosen to have good resistance to cell and nonspecific protein absorption, there should be minimum cell adhesion on the backing layer of the platform. For longer time periods (e.g., >2-3 weeks), different degrees of hydrogel degradation, foreign body reaction (presence of multinucleated foreign body giant cells), and granulation tissue formation may be observed.

Referring to FIG. 11, a system 1100 for screening multiple biological samples is shown. Platform 1102 can be provided on an optional translation stage 1104 for supporting the platform with respect to an evaluation device 1112 and for moving the platform with respect to the evaluation device 1112. The platform 1102 can be one or more of those discussed with respect FIGS. 1-9F above and formed according to the procedures disclosed elsewhere herein. When provided to the evaluation device 1112, the platform 1102 has undergone implantation and extraction from the host animal and is thus ready for evaluation of the biological samples contained thereon.

The evaluation device 1114 can include an optical system 1106, an imaging device 1108, and a processor 1110. The optical system 1106 can form an image of the biological samples of the platform 1102 for acquisition by the imaging device 1108. For example, the optical system 1106 may be a microscope (such as a light microscope, a confocal microscope, a fluorescence microscope, or any other microscope). The optical system 1106 may form an image of one biological sample at a time. Alternatively, the optical system 1106 may be configured to form an image of all of the biological samples at the same time. The imaging device 1108 can acquire and/or store the image provided by the optical system 1106. For example, the imaging device 1108 may be a charge-coupled device (CCD) or other digital camera, or a film camera. In embodiments, the platform is constructed such that the imaging device can acquire the necessary image or images without removal of the biological samples from the platform.

Processor 1110 can be configured to evaluate the image or images obtained by the imaging device 1108 and to determine characteristics of the biological samples based thereon. The determination of the processor 1110 may be communicated to, for example, to a user or an external system, via input/output 1112. The user and/or external system may also communicate with the processor 1110 and the rest of the evaluation device 1114 via input/output 1112, for example, to request evaluation of a particular testing region of the platform 1102.

For example, when the biological samples include materials for biocompatibility testing, the processor can be configured to determine inflammatory cell density on each biomaterial based on the images from the imaging device. Using conventional image processing techniques, the processor can identify inflammatory cells on each material surface, count the number of inflammatory cells, and then determine a normalized density based on a predetermined area of the material surface or by calculating the area from image. Alternatively or additionally, the processor 1110 can use image processing techniques to evaluate material degradation. The processor 1110 may be configured to set a biocompatibility score based on predetermined criteria. For example, those materials demonstrating an inflammatory cell density below a threshold may be characterized as good or acceptable biocompatibility.

When the biological samples include tumor spheroids for cancer drug testing, the processor can be configured to determine, based on the images from the imaging device, characteristics of each spheroid that relate to the effectiveness of the cancer drug thereon. For example, the processor can be configured to determine at least one of spheroid diameter, change in spheroid size, viable cell mass, percentage viability, and pathway activity. Using conventional image processing techniques, the processor can identify the spheroid in the platform and determine a size thereof. The size can then be compared with a previously determined size of the spheroid to determine if the tumor has grown, stayed the same, or reduced in size.

Systems, methods, and devices as described herein can thus provide high-throughput in vivo screening of biological samples. Biological samples are retained in microfabricated structures that allow precise, ordered placement of the samples both during and after implantation. In addition, the microfabricated platform allows host stroma cell interaction with a relatively large number (i.e., >20) of biological samples without direct interaction between the biological samples in the platform (i.e., no direct flowpath between the samples without passing into host stroma cell contact). In addition, platforms that include membranes can be designed with pore sizes that allow stroma cell infiltration in addition to molecular and soluble factor interaction for more realistic host microenvironment testing while still retaining tumor spheroids within the platform.

Although specific examples for use of the disclosed platform have been discussed herein, the disclosed platform is not limited to the study of a specific type of anti-cancer drug or material biocompatibility testing. Rather, teachings disclosed herein are useful for a variety of cell and material testing, including, but not limited to, all anti-cancer therapeutics that inhibit tumor cell growth by targeting tumor stroma, any drug therapy where the responses of different genotypes are expected to be different, and assessing host response to a range of other biomaterials (not just PEG).

In addition, although specific examples of the platform have been disclosed herein for biological samples arranged in a 2D array, it is contemplated that the samples could be arranged in a 3D array as well. For example, a pair of platform devices, such as the ones shown in FIGS. 2-3, could be bonded together after loading and culturing of the tumor spheroids to produce a 3D array. The membrane layer 202 of the first platform device would thus be on an opposite side of the bonded device from the membrane layer 202 of the second platform device.

In one or more embodiments, a method of culturing biological samples can include arranging biologically varied cells or tissue cultures in an array that spans a curvilinear planar region and exposing the varied cells or tissue cultures to living tissue of a host animal such that the cells or tissue cultures receive chemical signals from the living tissue. The method can further include regulating or perturbing the natural state of the host, and detecting an effect of the regulating or perturbing on the cells or tissue cultures. For example, the regulating or perturbing can include delivering a drug to the host animal. The curvilinear planar region can be a substantially flat planar region.

The arranging can include placing the varied cells or tissue cultures in a unitary structure having a respective compartment for each of the biologically varied cells or tissue cultures. Alternatively or additionally, the arranging can be such that an exposed face of the cells or tissue cultures face in a same direction.

The unitary structure can be configured to space the cells or tissue cultures less than 5 mm apart, for example, less than 1 mm apart. The unitary structure can have at least 20 compartments. The unitary structure can include microfluidic channels, each of which is connected to a respective one of the compartments and configured for loading the compartment. Each compartment of the unitary structure can have a diameter of 500 μm or less and a height of 300 μm or less. Each compartment can be isolated from the host animal by a membrane. Each membrane can be porous with a pore size to exclude the transfer of cytoplasmic bodies thereacross.

The exposing can include permitting the transmission of chemical signals across a membrane. Alternatively or additionally, the exposing can include surgically implanting the unitary structure in the host animal, for example, by surgically implanting the unitary structure in a stromal compartment of an organ of the host animal. The detecting can include surgically removing the unitary structure and performing an assay on the cells or tissue cultures in vitro while they are in the unitary structure.

It will be appreciated that the methods, processes, and systems described above can be implemented in hardware, hardware programmed by software, software instruction stored on a non-transitory computer readable medium or a combination of the above. For example, the processors described herein can be configured to execute a sequence of programmed instructions stored on a non-transitory computer readable medium. The processors can include, but are not limited to, a personal computer or workstation or other such computing system that includes a processor, microprocessor, microcontroller device, or is comprised of control logic including integrated circuits such as, for example, an Application Specific Integrated Circuit (ASIC). The instructions can be compiled from source code instructions provided in accordance with a programming language such as Java, C++, C#.net or the like. The instructions can also comprise code and data objects provided in accordance with, for example, the Visual Basic™ language, or another structured or object-oriented programming language. The sequence of programmed instructions and data associated therewith can be stored in a non-transitory computer-readable medium such as a computer memory or storage device which can be any suitable memory apparatus, such as, but not limited to read-only memory (ROM), programmable read-only memory (PROM), electrically erasable programmable read-only memory (EEPROM), random-access memory (RAM), flash memory, disk drive, etc.

Furthermore, the methods, processes, and systems can be implemented by a single processor or by a distributed processor. Further, it should be appreciated that the steps discussed herein can be performed on a single or distributed processor (single and/or multi-core). Also, the methods, processes, and systems described in the various figures of and for embodiments above can be distributed across multiple computers or systems or can be co-located in a single processor or system. Exemplary structural embodiment alternatives suitable for implementing the modules, sections, systems, means, or processes described herein are provided below, but not limited thereto.

The modules, processors or systems described herein can be implemented as a programmed general purpose computer, an electronic device programmed with microcode, a hard-wired analog logic circuit, software stored on a computer-readable medium or signal, an optical computing device, a networked system of electronic and/or optical devices, a special purpose computing device, an integrated circuit device, a semiconductor chip, and a software module or object stored on a computer-readable medium or signal, for example. Moreover, embodiments of the disclosed methods, processes, systems, and computer program product can be implemented in software executed on a programmed general purpose computer, a special purpose computer, a microprocessor, or the like.

Embodiments of the methods and systems (or their sub-components or modules), can be implemented on a general-purpose computer, a special-purpose computer, a programmed microprocessor or microcontroller and peripheral integrated circuit element, an ASIC or other integrated circuit, a digital signal processor, a hardwired electronic or logic circuit such as a discrete element circuit, a programmed logic circuit such as a programmable logic device (PLD), programmable logic array (PLA), field-programmable gate array (FPGA), programmable array logic (PAL) device, etc. In general, any process capable of implementing the functions or steps described herein can be used to implement embodiments of the method, system, or a computer program product (software program stored on a non-transitory computer readable medium).

Furthermore, embodiments of the disclosed methods, processes, systems, and computer program products can be readily implemented, fully or partially, in software using, for example, object or object-oriented software development environments that provide portable source code that can be used on a variety of computer platforms. Alternatively, embodiments of the disclosed methods, processes, systems, and computer program products can be implemented partially or fully in hardware using, for example, standard logic circuits or a very-large-scale integration (VLSI) design. Other hardware or software can be used to implement embodiments depending on the speed and/or efficiency requirements of the systems, the particular function, and/or particular software or hardware system, microprocessor, or microcomputer being utilized. Embodiments of the disclosed methods, processes, systems, and computer program products can be implemented in hardware and/or software using any known or later developed systems or structures, devices and/or software by those of ordinary skill in the applicable art from the function description provided herein and with a general basic knowledge of imaging and/or computer programming arts.

As mentioned above, aspects of the disclosed subject matter may be embodied in the form a platform that, once implanted, remains within, and is assayed within, the host. For example, engineered tissue platform embodiments may provide a suitable format for a multiplexed array of genotypes to be inserted in the body. The host animal may be opened and one or more labeling devices may be applied to the platform permitting the cells to be assayed in situ without removing the platform from the host. Alternatively or additionally, the platform may be provided with additional interrogation components (e.g., light source and detector) that can provide for in situ assay without opening the host animal. Alternatively or additionally, a separate assaying device can be implanted in the host animal (at a same time or a later time as the platform) in order to provide for in situ assay of the platform.

Furthermore, the foregoing descriptions apply, in some cases, to examples generated in a laboratory, but these examples can be extended to production techniques. For example, where quantities and techniques apply to the laboratory examples, they should not be understood as limiting. In addition, although specific chemicals and materials have been disclosed herein, other chemicals and materials may also be employed according to one or more contemplated embodiments.

Features of the disclosed embodiments may be combined, rearranged, omitted, etc., within the scope of the invention to produce additional embodiments. Furthermore, certain features may sometimes be used to advantage without a corresponding use of other features.

It is thus apparent that there is provided in accordance with the present disclosure, system, methods, and devices for multiplexed in vivo screening of biological samples. Many alternatives, modifications, and variations are enabled by the present disclosure. While specific embodiments have been shown and described in detail to illustrate the application of the principles of the present invention, it will be understood that the invention may be embodied otherwise without departing from such principles. Accordingly, Applicants intend to embrace all such alternatives, modifications, equivalents, and variations that are within the spirit and scope of the present invention.

1-13. (canceled)

14. A device for screening multiple biological samples in vivo, the device comprising:
a platform member having a plurality of testing regions thereon, each of the testing regions being configured to hold a different biological sample for interaction in vivo with stroma cells when implanted into a host animal,
the platform member including isolating portions arranged between adjacent testing regions such that fluid and/or material cannot pass from one testing region to another testing region without contacting the host animal stroma cells when implanted into the host animal.

15. The device of claim 14, wherein the testing regions are arranged in a two-dimensional array with isolating portions arranged therebetween.

16. The device of claim 14, wherein the testing regions and isolating portions are arranged such that crosstalk between testing regions is inhibited.

17. (canceled)

18. The device of claim 14, wherein each testing region includes a chamber with a membrane layer, the membrane layer being constructed to retain a tumor spheroid within said chamber while allowing interaction between the tumor spheroid and host cell stroma through the membrane layer.

19. The device of claim 18, wherein the platform includes a plurality of separate microfluidic channels, each channel being connected to a respective chamber.

20. The device of claim 14, wherein each testing region includes a block of a biomaterial to be tested and the isolating portions include a biocompatible hydrogel-based backing layer.

21. A system for screening multiple biological samples in vivo, the system comprising:
a screening device including a platform with a plurality of testing regions thereon, each of the testing regions being configured to hold a different biological sample for interaction in vivo with stroma cells when implanted into a host animal, the platform further include isolating portions arranged between adjacent testing regions such that fluid and/or material can only pass from one testing region to another testing region by contacting the host animal stroma cells when implanted into the host animal; and
an evaluation device configured to image the testing regions ex vivo so as to determine the effect of the in vivo exposure on the biological samples in the platform.

22. The system of claim 21, wherein the evaluation device includes an imaging device configured to acquire an image of each biological sample, and the platform is constructed such that the biological samples can be imaged by the imaging device in situ.

23. The system of claim 22, wherein the biological samples include biomaterials for biocompatibility testing, and the evaluation device includes a processor configured to determine inflammatory cell density on each biomaterial based on the images from the imaging device.

24. The system of claim 22, wherein the biological samples include tumor spheroids of different genotypes, and the evaluation device includes a processor configured to determine for each tumor spheroid at least one of spheroid diameter, change in spheroid size, viable cell mass, percentage viability, and pathway activity based on the images from the imaging device.

25. A method of culturing biological samples, comprising:
arranging biologically varied cells or tissue cultures in an array that spans a curvilinear planar region;
exposing the varied cells or tissue cultures to living tissue of a host animal such that the cells or tissue cultures receive chemical signals from the living tissue;
regulating or perturbing the natural state of the host; and
detecting an effect of the regulating or perturbing on the cells or tissue cultures.

26. The method of claim 25, wherein the regulating or perturbing includes delivering a drug to the host animal.

27. The method of claim 25, wherein the arranging includes placing the varied cells or tissue cultures in a unitary structure having a respective compartment for each of the biologically varied cells or tissue cultures.

28. (canceled)

29. The method of claim 27, wherein the unitary structure is configured to space the cells or tissue cultures less than 1 mm apart.

30. The method of claim 27, wherein the exposing includes surgically implanting the unitary structure in the host animal.

31. (canceled)

32. The method of claim 30, wherein the detecting includes surgically removing the unitary structure and performing an assay on the cells or tissue cultures in vitro while they are in the unitary structure.

33-36. (canceled)

37. The method of claim 27, wherein the unitary structure has at least 20 compartments.

38. The method of claim 25, wherein the curvilinear planar region is a substantially flat planar region.

39. The method of claim 25, wherein the exposing includes permitting the transmission of chemical signals across a membrane.

40. The method of claim 25, wherein the arranging is such that an exposed face of the cells or tissue cultures face in a same direction.

41-46. (canceled)
